SEQUENCE LISTING (12 sequences)

SEQ ID NO: 1 - protein, 9 aa, Apostichopus japonicus
CFITNCPLG

SEQ ID NO: 2 - protein, 9 aa, Homo sapiens
CYIQNCPLG

SEQ ID NO: 3 - protein, 9 aa, Homo sapiens
CYFQNCPRG

SEQ ID NO: 4 - protein, 9 aa, Octopus vulgaris
CFWTNCPVG

SEQ ID NO: 5 - RNA, 20 nt, Homo sapiens
  source 1..20: mol_type = genomic RNA
caagatgcacaactcggaga

SEQ ID NO: 6 - RNA, 20 nt, Homo sapiens
  source 1..20: mol_type = genomic RNA
ttcatgtgcgcgtaactgtc

SEQ ID NO: 7 - RNA, 20 nt, Homo sapiens
  source 1..20: mol_type = genomic RNA
tgggatatacacaggccgat

SEQ ID NO: 8 - RNA, 20 nt, Homo sapiens
  source 1..20: mol_type = genomic RNA
gtgacagagacagggggaaa

SEQ ID NO: 9 - RNA, 20 nt, Homo sapiens
  source 1..20: mol_type = genomic RNA
gacagtcagccgcatcttct

SEQ ID NO: 10 - RNA, 20 nt, Homo sapiens
  source 1..20: mol_type = genomic RNA
gcgcccaatacgaccaaatc

SEQ ID NO: 11 - RNA, 20 nt, Homo sapiens
  source 1..20: mol_type = genomic RNA
tgatgcaatctggcacttcg

SEQ ID NO: 12 - RNA, 20 nt, Homo sapiens
  source 1..20: mol_type = genomic RNA
gccagcacacacacgataag

(19) United States
(12) Patent Application Publication
(10) Pub. No.: US 2026/0209286 A1
(43) Pub. Date: Jul. 23, 2026

(54) SEA CUCUMBER-DERIVED HOLOTOCIN PEPTIDE AND USES THEREOF

(71) Applicant: NATIONAL MARINE BIODIVERSITY INSTITUTE OF KOREA, Chungcheongnam-do (KR)

(72) Inventors: Jei Ha Lee, Daejeon (KR); Seon Mi Jo, Jeollabuk-do (KR); Seung Hyun Jung, Chungcheongnam-do (KR); Hea Bin Kim, Jeollabuk-do (KR)

(21) Appl. No.: 19/139,032

(22) PCT Filed: Dec. 15, 2023
(86) PCT No.: PCT/KR2023/020737

§ 371 Date: Jun. 13, 2025

(30) Foreign Application Priority Data
Dec. 15, 2022 (KR) 10-2022-0175701
Dec. 15, 2023 (KR) 10-2023-0182721

(51) Int. Cl. C07K 14/435; A61K 8/64; A61K 38/00; A61P 17/02; A61Q 19/00; A61Q 19/08

(52) U.S. Cl. CPC C07K 14/43504; A61K 8/64; A61P 17/02; A61Q 19/007; A61Q 19/08; A61K 38/00

(57) ABSTRACT

The present invention relates to a composition for promoting the formation of stem cell spheroids, including sea cucumber-derived holotoxin peptide. When applied to cells, the composition for promoting the formation of stem cell spheroids, including sea cucumber-derived holotocin peptide according to the present invention, may promote the formation of three-dimensional spheroids and enhance the expression of cell regeneration-related genes, thereby contributing to the formation of new biological tissues or the regeneration of damaged or aged tissues. Therefore, it can be utilized in cosmetic compositions, pharmaceuticals, quasi-drugs, health functional foods, veterinary drugs, or a feed compositions for promoting wound healing, regeneration, skin moisturization, and anti-aging in the skin and other body tissues of humans or animals.

### TECHNICAL FIELD

The present invention relates to a composition for promoting the formation of stem cell spheroids, comprising a sea cucumber-derived holotocin peptide. More specifically, the present invention relates to a composition for promoting the formation of stem cell spheroids, which promotes the formation of spheroids and exhibits cell regeneration effects in a manner similar to human oxytocin or vasopressin, by utilizing a holotocin peptide derived from a natural marine organism.

### BACKGROUND ART

Stem cells have multipotent differentiation potential, meaning that they are capable of differentiating into various specific cell types, and thus have potential for use in cell therapy, particularly due to their ability to differentiate into cells at sites of tissue damage. However, in actual clinical applications, it is difficult to find cases of broad and stable success, due to difficulties in securing appropriate stem cells and low survival rates after in vivo transplantation.

The present inventors, while continuing their research on regenerative compositions capable of promoting renal stem cell differentiation by increasing the formation and size of spheroids during the cultivation of spheroids derived from renal cells, paid attention to literature reporting that vasopressin (arginine vasopressin; AVP) and oxytocin increase the size of spheroids in rat hypothalamic neurons (Salehi, M. S., Neumann, I. D., Jurek, B., & Pandamooz, S. (2021). Co-stimulation of oxytocin and arginine-vasopressin receptors affect hypothalamic Neurospheroid size. International Journal of Molecular Sciences, 22 (16), 8464).

Vasopressin (arginine vasopressin; AVP) and oxytocin, which are found in humans, are peptide hormones and antidiuretic hormones secreted from the posterior pituitary gland, and are also involved in the expression of aquaporin proteins.

According to recent research, oxytocin has been found to induce reprogramming of mature epicardial cells into stem cells, which subsequently migrate to the myocardium, which is the deep layer of the damaged heart, where they secrete therapeutic substances and differentiate into vascular cells or cardiomyocytes.

Accordingly, attention was directed to octopressin, a peptide derived from Octopus minor and known as a hormone similar to oxytocin, and an experiment was conducted to compare octopressin, vasopressin, and oxytocin in order to discover regeneration-promoting substances.

Moreover, inspired by the regenerative ability of sea cucumbers, which expel their internal organs to escape when threatened and later regenerate the lost organs, a search for useful genes was conducted based on genome and transcriptome analysis of Stichopus japonicus Selenka in order to identify regeneration-promoting substances.

The present inventors, while continuing their research on regeneration-promoting substances using octopressin, vasopressin, and oxytocin, further incorporated holotocin extracted from Stichopus japonicus Selenka. As a result of this research, they found that holotocin promotes the formation of stem cell spheroids and exhibits excellent cell regeneration effects, thereby arriving at the present invention.

### DISCLOSURE

### Technical Problem

The present invention relates to a composition for promoting the formation of stem cell spheroids, comprising a sea cucumber-derived holotocin peptide. Moreover, the present invention aims to provide a composition that promotes the formation of stem cell spheroids and exhibits cell regeneration effects without inducing cytotoxicity, thereby enabling wound healing, regeneration, skin moisturization, or anti-aging in the skin or other body tissues.

However, the technical problems to be solved by the present invention are not limited to those described above, and other problems not specifically mentioned will be clearly understood by those skilled in the art from the following description.

### Technical Solution

According to one embodiment of the present invention, there is provided a composition for promoting the formation of stem cell spheroids, comprising a sea cucumber-derived holotocin peptide.

In the present invention, the sea cucumber may be Stichopus japonicus Selenka.

In the present invention, the sea cucumber-derived holotocin peptide may comprise the amino acid sequence of SEQ ID NO: 1.

In the present invention, the sea cucumber-derived holotocin peptide may increase the size of three-dimensional cell spheroids by 105% to 130% during cell culture.

In the present invention, the sea cucumber-derived holotocin peptide may increase the expression of at least one gene or protein selected from the group consisting of Sox2 (sex determining region Y-box 2), Oct4 (octamer-binding transcription factor 4), and AQP3 (Aquaporin 3).

In the present invention, the sea cucumber-derived holotocin peptide may promote the regeneration of at least one cell selected from the group consisting of stem cells, hepatocytes, skin cells, muscle cells, fibroblasts, and nerve cells.

In the present invention, the composition for promoting the formation of stem cell spheroids may have the function of cell regeneration, skin regeneration, skin wound healing, skin barrier improvement, skin moisturization, or anti-aging.

According to another embodiment of the present invention, there is provided a method for promoting the formation of stem cell spheroids, comprising the step of treating cells with a sea cucumber-derived holotocin peptide.

According to still another embodiment of the present invention, there is provided a pharmaceutical composition for cell regeneration or wound healing, comprising the composition for promoting the formation of stem cell spheroids prepared according to the present invention.

According to yet another embodiment of the present invention, there is provided a cosmetic composition for skin cell regeneration, moisturization, or anti-aging, comprising the composition for promoting the formation of stem cell spheroids prepared according to the present invention.

According to still yet another embodiment of the present invention, there is provided a topical composition for cell regeneration or wound healing, comprising the composition for promoting the formation of stem cell spheroids prepared according to the present invention.

According to a further embodiment of the present invention, there is provided a quasi-drug composition for cell regeneration or wound healing, comprising the composition for promoting the formation of stem cell spheroids prepared according to the present invention.

According to another further embodiment of the present invention, there is provided a health functional food for cell regeneration or anti-aging, comprising the composition for promoting the formation of stem cell spheroids prepared according to the present invention.

According to a still further embodiment of the present invention, there is provided a feed composition for cell regeneration or anti-aging, comprising the composition for promoting the formation of stem cell spheroids prepared according to the present invention.

### Advantageous Effects

The composition for promoting the formation of stem cell spheroids, comprising the sea cucumber-derived holotocin peptide according to the present invention, promotes the formation of three-dimensional spheroids, thereby facilitating stem cell differentiation and enhancing the expression of cell regeneration-related genes, ultimately exhibiting excellent effects on the regeneration of biological tissues.

Therefore, it can be utilized in cosmetic compositions, pharmaceuticals, quasi-drugs, health functional foods, veterinary drugs, or feed compositions for promoting wound healing, regeneration, skin moisturization, or anti-aging in the skin or other body tissues of humans or animals.

### BRIEF DESCRIPTION OF DRAWINGS

FIGS. 1 to 4 relate to Example 1 and show the results of cytotoxicity evaluation of marine peptides in 293T and HaCaT cells.

FIGS. 5 to 12 relate to Example 2 and show the results of evaluating the size of stem cell spheroids formed in 293T and HaCaT cells in response to holotocin.

FIGS. 13 and 14 relate to Example 3 and show the expression of regeneration-related genes in HaCaT cells in response to holotocin.

FIGS. 15 and 16 relate to Example 4 and show the expression of the AQP3 gene in HaCaT cells in response to holotocin.

### BEST MODE FOR CARRYING OUT THE INVENTION

Provided are: a composition for promoting the formation of stem cell spheroids, comprising a sea cucumber-derived holotocin peptide; a cosmetic composition for skin cell regeneration, moisturization, or anti-aging, comprising the same; and a pharmaceutical composition, topical composition, quasi-drug composition, health functional food, veterinary drug, or feed composition for cell regeneration or wound healing, all comprising the above-mentioned composition.

### MODE FOR CARRYING OUT THE INVENTION

Hereinafter, the present invention will be described in more detail with reference to the following embodiments. However, the following embodiments are provided for illustrative purposes only and are not intended to limit the scope of the present invention.

According to one embodiment of the present invention, there is provided a composition for promoting the formation of stem cell spheroids, comprising a sea cucumber-derived holotocin peptide.

“Sea cucumbers” are marine invertebrates belonging to the class Holothuroidea of the phylum Echinodermata. In Korea, four families and fourteen species are known to inhabit the coastal waters, including green, black, and red sea cucumbers. Sea cucumbers, along with ginseng, have long been regarded as representative health-promoting foods and have been the subject of nutritional research from ancient times to the present. Traditional medical texts such as Jasaneobo, Bonchogangmok, and Donguibogam describe their effects on restoring vitality and calming the nerves, indicating their widespread historical use. Recent studies have revealed that sea cucumbers have remarkable biological regenerative abilities, and as a result, they are now widely cultivated as a valuable species with high nutritional and medicinal value.

In particular, although the efficacy of sea cucumbers in promoting skin cell regeneration has been demonstrated, especially as a natural product-based therapeutic agent, most studies on skin regeneration and whitening have involved the combined use of extracts derived from sea cucumbers and other materials. Therefore, it has been difficult to clearly determine the efficacy of sea cucumbers alone in promoting skin regeneration and whitening. Moreover, in studies that investigated sea cucumbers alone, there have been no detailed reports on how differences in species, body parts, or extraction methods affect their skin-related bioactivity.

In the present invention, the sea cucumber may be Stichopus japonicus Selenka, and the Stichopus japonicus Selenka has a cylindrical body elongated in the anterior-posterior direction and is characterized by multiple wart-like protuberances on its dorsal surface. The Polian vesicle of the sea cucumber is slender and elongated with a pointed tip, and its eggs are enveloped in a gelatinous membrane approximately 25 μm in thickness. In particular, it exhibits high contractility and exceptional regenerative capacity, contributing to excellent wound-healing properties in damaged areas.

Furthermore, commonly used sea cucumbers such as blue sea cucumber, black sea cucumber, red sea cucumber, white sea cucumber, or jellyfish sea cucumber may be used. Preferably, the red sea cucumber may be used from the standpoint of utilizing Stichopus japonicus Selenka, which is rich in active ingredients such as holotocin, chondroitin, collagen, taurine, and alginic acid.

In the present invention, the sea cucumber-derived holotocin is an oxytocin/vasopressin family peptide derived from Apostichopus japonicas, a species belonging to the class Holothuroidea of the phylum Echinodermata. It is predicted to have a structure in which the first and sixth cysteine residues are linked by a disulfide bond, and the C-terminal glycine is amidated. In addition, it has been reported that short-term injection of holotocin into sea cucumbers causes body swelling, whereas long-term injection may contribute to a reduction in body mass through mechanisms such as osmotic regulation.

In the present invention, the sea cucumber-derived holotocin peptide may comprise the amino acid sequence of SEQ ID NO: 1.
CFITNCPLG-NH2 (SEQ ID NO: 1)

In the present invention, the sea cucumber-derived holotocin peptide may promote the formation of three-dimensional cell spheroids during cell culture. When cells are treated with the sea cucumber-derived holotocin peptide, the formation of three-dimensional cell spheroids is promoted, thereby inducing or promoting the differentiation of stem cells.

Specifically, the sea cucumber-derived holotocin peptide may increase the size of three-dimensional cell spheroids by 115% to 130% during cell culture.

The term “spheroid” refers to a three-dimensional spherical aggregate consisting of more than 1,000 individual cells. Such spheroids can more accurately mimic the structural and physical properties of the three-dimensional tissues that surround cells in the human body, and thus can be effectively utilized in therapeutic and research applications.

In particular, three-dimensional culture, in which cells are spatially arranged and cultured in a test tube, allows the cells to grow in all directions in vitro, unlike two-dimensional culture. This enables the creation of an environment that more closely mimics in vivo cellular conditions, thereby facilitating cell differentiation through the formation of three-dimensional spheroids.

Accordingly, the three-dimensional spheroid may refer to a three-dimensional spheroid-type cell aggregate.

The size of the three-dimensional spheroids may range from 50 μm to 600 μm, preferably from 70 μm to 550 μm, and more preferably from 100 μm to 500 μm, but is not limited thereto.

For example, if the size of the three-dimensional spheroids is less than 50 μm, it may be too small to handle easily. On the other hand, if it exceeds 600 μm, the diffusion of gases and nutrients under culture conditions may become insufficient, resulting in an increase in cell death, which can lead to insufficient therapeutic efficacy when the spheroids are transplanted into target cells or tissues.

In the present invention, the sea cucumber-derived holotocin peptide may increase the expression of at least one gene or protein selected from the group consisting of Sox2 (sex determining region Y-box 2), Oct4 (octamer-binding transcription factor 4), and AQP3 (Aquaporin 3).

The Sox2 (sex determining region Y-box 2) and Oct4 (octamer-binding transcription factor 4) are key transcription factors that determine pluripotency. They upregulate genes associated with stemness and suppress genes involved in differentiation, thereby maintaining or enhancing the ability of stem cells to form spheroids.

The AQP3 (Aquaporin 3) is a type of aquaporin protein.

Aquaporins (AQPs) are integral membrane proteins that form channels in the cell membrane to facilitate the passive transport of water molecules. Discovered in 1992, aquaporins act as selective pores that allow the transport of water, glycerol, and small solutes across the cell membrane, and are widely expressed in both plants and animals.

In addition, aquaporins are specifically distributed depending on the type of cell or tissue, and are present in the cell membrane as tetramers, each composed of six α-helices as basic structural units that span the cell membrane. These subunits function cooperatively and are involved in various physiological processes, including urine formation, secretion of digestive fluids, protection against dehydration, and even cell-to-cell adhesion.

Aquaporins are broadly classified into approximately 13 subtypes, and 13 types (AQP0-AQP12) have been identified in humans. Among them, aquaporin 3 is the subtype most closely associated with the skin. It is expressed in the keratinocytes of both mice and humans and is present from the basal layer to the granular layer, but disappears in the stratum corneum. That is, aquaporin 3 is known to be the most abundantly expressed in the skin and plays key roles in moisturization, wound healing, and the maintenance of epidermal homeostasis. Specifically, it was first genetically cloned from the kidney of a mouse, and its role in water transport was confirmed by the observation that osmotic water transport in the collecting ducts was reduced by approximately threefold in AQP3-deficient mice. Moreover, it is known to be expressed on the cell membrane, where it facilitates the uptake of water molecules from the intercellular space and serves as a channel that distributes water evenly throughout the cell.

Therefore, skin lacking aquaporin 3 exhibits reduced elasticity and impaired skin barrier recovery (Boury-Jamot M, Handb Exp Pharmacol., 2009 (190):205-217)).

Furthermore, it facilitates the transport of glycerol across the cell membrane and shares a similar amino acid sequence and protein structure with GlpF, a glycerol transporter in Escherichia coli. Therefore, increased expression of AQP3 leads to higher moisture content in skin tissues, thereby contributing to a more youthful appearance and enhanced skin elasticity.

In addition, it has a significant effect on the healing of skin damage such as roughness and enhances cell regeneration or wound healing capabilities.

That is, the sea cucumber-derived holotocin peptide can increase the expression of Sox2, Oct4, and AQP3 proteins in skin cells, thereby enhancing the effect of skin cell regeneration, skin barrier function enhancement, skin moisturization, or anti-aging.

In the present invention, the sea cucumber-derived holotocin peptide may promote the regeneration of at least one cell selected from the group consisting of stem cells, hepatocytes, skin cells, muscle cells, fibroblasts, and nerve cells.

For example, it can promote cell growth, thereby enabling cell regeneration and supporting the formation or regeneration of biological tissues. As a result, it may be effective not only in wound healing and regeneration in the skin or other body tissues, but also in skin moisturization or anti-aging.

In the present invention, the stem cell may be at least one selected from the group consisting of adult stem cells, embryonic stem cells, mesenchymal stem cells, adipose stem cells, hematopoietic stem cells, human induced pluripotent stem cells, umbilical cord blood stem cells, and dedifferentiated stem cells.

The term “stem cell” refers to an undifferentiated cell that has the potential to differentiate but has not yet undergone differentiation. Under appropriate conditions, such undifferentiated stem cells can differentiate into various types of tissue cells, and research utilizing this property for disease treatment and other applications is actively ongoing.

To utilize stem cells, it is necessary to maintain their stemness until they are differentiated into specific cell types for application. To this end, three-dimensional culture systems are being investigated for the preservation of stemness, and stem cells in the form of three-dimensional spheroids may be employed.

In the present invention, the composition for promoting the formation of stem cell spheroids may have the function of cell regeneration, skin regeneration, skin wound healing, skin barrier improvement, skin moisturization, or anti-aging. Accordingly, it can be utilized in cosmetic compositions, pharmaceuticals, quasi-drugs, health functional foods, veterinary drugs, or feed compositions for promoting wound healing, regeneration, skin moisturization, or anti-aging in the skin or other body tissues of humans or animals.

According to another embodiment of the present invention, there is provided a method for promoting the formation of stem cell spheroids, comprising the step of treating cells with a sea cucumber-derived holotocin peptide.

In the present invention, the cell may be at least one selected from the group consisting of stem cells, hepatocytes, skin cells, muscle cells, fibroblasts, and nerve cells, and may be skin cells in terms of achieving the intended effect.

The cells may be treated with the sea cucumber-derived holotocin peptide at a concentration of 0.01 μM or higher, and at 1 μM or higher to promote cell regeneration and the expression of target genes. Moreover, since no cytotoxicity is observed even at higher concentrations, the cells may be treated with various concentrations depending on the intended effect.

In the present invention, the size of the three-dimensional cell spheroids treated with the sea cucumber-derived holotocin peptide may be increased by 105% to 130%.

For example, the cells may be treated with the sea cucumber-derived holotocin peptide at a concentration of 0.01 to 15 μM, 0.05 to 13 μM, or 0.07 to 11 μM, and more preferably at a concentration of 0.1 to 10 μM to increase the size of the three-dimensional cell spheroids by 105% to 130% compared to that before the treatment.

More specifically, the size of the spheroids may be increased by 115% to 130% when renal cells are treated with the sea cucumber-derived holotocin peptide at a concentration of 0.05 to 10 μM. Similarly, the size of the spheroids may be increased by 115% to 130% when skin cells are treated with the sea cucumber-derived holotocin peptide at a concentration of 0.05 to 3 μM.

In the present invention, the cells treated with the sea cucumber-derived holotocin peptide may exhibit an increased expression of at least one gene or protein selected from the group consisting of Sox2, Oct4, and AQP3. The increased expression of Sox2 and Oct4 may enhance the effect of cell regeneration or regeneration promotion, while the increased expression of the AQP3 gene may enhance the effect of wound healing, regeneration, skin moisturization, or anti-aging in the skin or other body tissues.

According to still another embodiment of the present invention, there is provided a pharmaceutical composition for cell regeneration or wound healing, comprising the composition for promoting the formation of stem cell spheroids prepared according to the present invention.

In the present invention, the pharmaceutical composition may comprise at least one pharmaceutically acceptable excipient, particularly a dermatologically acceptable excipient.

Furthermore, the pharmaceutical composition of the present invention may be used as a cell therapy product. The term “cell therapy product” refers to a pharmaceutical product (as defined by the U.S. FDA) used for therapeutic, diagnostic, or preventive purposes, which is prepared from cells and tissues that have been isolated, cultured, and specifically manipulated from humans. Specifically, it refers to a pharmaceutical product used for therapeutic, diagnostic, or preventive purposes through a series of actions such as ex vivo proliferation and selection of living autologous, allogeneic, or xenogeneic cells, or by otherwise altering the biological characteristics of the cells to restore the functions of cells or tissues.

The term “prevention” refers to any action that inhibits or delays cell regeneration, while the term “improvement” or “treatment” refers to any action that beneficially alters cells by promoting cell regeneration through administration or application of the composition.

For example, a person having ordinary skill in the art to which the present invention pertains would be able to determine the exact criteria for diseases for which the composition of the present invention is effective, and to assess the degree of improvement, enhancement, or treatment, with reference to data provided by organizations such as the Korean Medical Association. In addition, the pharmaceutical composition may comprise the active ingredient alone or in combination with one or more pharmaceutically acceptable carriers, excipients, or diluents. Specific examples thereof may include lactose, dextrose, sucrose, sorbitol, mannitol, xylitol, erythritol, maltitol, starch, acacia gum, alginate, gelatin, calcium phosphate, calcium silicate, cellulose, methylcellulose, microcrystalline cellulose, polyvinylpyrrolidone, water, methylhydroxybenzoate, propylhydroxybenzoate, talc, magnesium stearate, and mineral oil.

As used herein, the term “pharmaceutically acceptable” refers to a composition that is physiologically acceptable and does not typically cause any allergic or similar reaction when administered to humans.

Furthermore, the pharmaceutical composition may be provided in combination with a conventionally known therapeutic agent for skin cell protection or skin regeneration. That is, the pharmaceutical composition may be administered in combination with a known compound that has the effects of skin regeneration and treatment.

The term “administration” refers to the introduction of a given substance into a subject by an appropriate method, and the term “subject” refers to any animal including humans, rats, mice, and livestock, that is in need of cell regeneration, skin wound healing, or skin barrier improvement. A specific example may be a mammal including a human.

If necessary, the pharmaceutical composition may further comprise a compound for the treatment of damaged skin or for cell regeneration.

The administration route of the pharmaceutical composition may include, but is not limited to, oral, intravenous, intramuscular, intraarterial, intramedullary, intrathecal, intracardiac, transdermal, subcutaneous, intraperitoneal, intranasal, enteral, topical, sublingual, or rectal administration.

The pharmaceutical composition may be administered either orally or parenterally. In the case of parenteral administration, it is preferably administered via topical application to the skin, intraperitoneal injection, intrarectal injection, subcutaneous injection, intravenous injection, intramuscular injection, or intrathoracic injection, but is not limited thereto. Oral administration may be preferred in terms of achieving more effective absorption.

In the case of oral formulations, the composition of the present invention may be formulated into dosage forms such as powders, granules, tablets, pills, dragees, capsules, liquids, gels, syrups, slurries, or suspensions by methods known in the art. For example, the oral formulations may be prepared in the form of tablets or dragees by mixing the active ingredient with solid excipients, grinding the mixture, adding suitable additives, and granulating the resulting mixture. Examples of suitable excipients may include sugars, including lactose, dextrose, sucrose, sorbitol, mannitol, xylitol, erythritol, and maltitol; starches, including corn starch, wheat starch, rice starch, and potato starch; celluloses, including cellulose, methylcellulose, sodium carboxymethyl cellulose, and hydroxypropylmethyl-cellulose; and fillers, such as gelatin and polyvinylpyrrolidone. Moreover, disintegrants such as cross-linked polyvinylpyrrolidone, agar, alginic acid, or sodium alginate may be added, if necessary. Furthermore, the pharmaceutical composition of the present invention may further comprise anti-agglomerating agents, lubricants, wetting agents, flavoring agents, emulsifiers, and preservatives.

In the case of parenteral formulations, the composition of the present invention may be formulated in the form of injections, creams, lotions, topical ointments, oils, moisturizers, gels, aerosols or nasal inhalers by methods known in the art. These formulations are described in the standard reference commonly used in the field of pharmaceutical chemistry (Remington's Pharmaceutical Sciences, 15^{th} Edition, 1975, Mack Publishing Company, Easton, Pennsylvania 18042, Chapter 87: Blaug, Seymour).

The preferred dosage of the pharmaceutical composition may vary depending on the patient's condition and weight, the severity of the disease, the dosage form, and the route and duration of administration, but can be appropriately selected by those skilled in the art. However, to achieve desirable effects, the content of the active ingredient in the pharmaceutical composition of the present invention may be adjusted according to the severity of the disease. The preferred total dosages of the pharmaceutical composition of the present invention may vary depending on factors such as the patient's weight, age, sex, health condition, diet, duration and route of administration, excretion rate, and severity of disease. However, the effective daily dosage for an adult (60 kg) is typically about 1 to 100 g/day, preferably about 10 to 50 g/day, and more preferably about 30 g/day. Since the dosage may vary depending on various conditions, it will be apparent to those skilled in the art that the above-described dosage may be increased or decreased. Accordingly, the dosage should not be construed as limiting the scope of the present invention in any way.

The number of administrations may be once or several times a day within a desired range, and the duration of administration is not particularly limited. Furthermore, the pharmaceutical composition of the present invention may be administered orally as is, or may be added to any type of food for routine intake. In view of the foregoing, a person having ordinary skill in the art will be able to determine an appropriate effective dosage according to the specific use. The pharmaceutical composition according to the present invention is not particularly limited in its formulation, route of administration, and method of administration, as long as it exhibits the effects of the present invention. When formulated, the pharmaceutical composition may be prepared using commonly used diluents or excipients such as fillers, extenders, binders, wetting agents, disintegrants, and surfactants. Solid formulations for oral administration may include tablets, pills, powders, granules, and capsules, and such solid formulations may be prepared by mixing the composition with at least one excipient, such as starch, calcium carbonate, sucrose or lactose, or gelatin. In addition to simple excipients, lubricants such as magnesium stearate and talc may also be used. Liquid formulations for oral administration may include suspensions, internal solutions, emulsions, and syrups, and may contain not only commonly used simple diluents such as water and liquid paraffin, but also various excipients such as wetting agents, sweeteners, flavoring agents, and preservatives. Formulations for parenteral administration may include sterile aqueous solutions, non-aqueous solvents, suspensions, emulsions, lyophilized formulations, and suppositories. Non-aqueous solvents and suspensions may include propylene glycol, polyethylene glycol, vegetable oils such as olive oil, and injectable esters such as ethyl oleate. Suppository bases may include Witepsol, macrogol, Tween 61, cacao butter, laurin butter, and glycerogelatin.

Furthermore, the pharmaceutical composition may further comprise anti-agglomerating agents, lubricants, wetting agents, flavoring agents, emulsifiers, and preservatives.

In the case of parenteral formulations, the composition of the present invention may be formulated in the form of injections by methods known in the art, and these formulations are described in the standard reference commonly used in the field of pharmaceutical chemistry (Remington's Pharmaceutical Sciences, 15^{th} Edition, 1975, Mack Publishing Company, Easton, Pennsylvania 18042, Chapter 87: Blaug, Seymour).

The feed composition according to the present invention may be applied without limitation to any subject for the purpose of enhancing immune function or exhibiting anticancer activity. For example, it may be applied to any subject, including non-human animals such as monkeys, dogs, cats, rabbits, guinea pigs, rats, mice, cattle, sheep, pigs, and goats, birds, and fish.

In view of the complexity of the present specification, redundant content is omitted. Unless otherwise defined herein, the terms used in the present specification shall be interpreted as having meanings commonly understood by those skilled in the art to which the present invention pertains.

Next, the present invention will be described in more detail with reference to the following Examples. It will be apparent to those skilled in the art that these Examples are provided merely to illustrate the present invention, and the scope of the present invention is not to be construed as being limited thereby.

### EXAMPLES

### Preparation Example 1: Preparation of Marine Peptides

Peptides belonging to the oxytocin/vasopressin superfamily may exhibit significantly different functions even with only one or two amino acid sequence differences. Accordingly, to investigate the cell regeneration or regeneration-promoting substances of marine-derived peptides, including such peptides, holotocin (HLT), oxytocin (OXT), vasopressin (AVP), and octopressin (OTP) were prepared.

Specifically, the sequences of holotocin (MF401997), oxytocin (M25650), and vasopressin (M25647) were identified using gene information from the National Center for Biotechnology Information (NCBI), and the information on octopressin was referenced from the active peptide of Octopus minor octopressin and uses thereof, as disclosed by the National Marine Biodiversity Institute of Korea.

Accordingly, peptides having the sequences listed in Table 1 below, each with a purity of 98% or higher, were synthesized by commissioning Anygen Co., Ltd.

TABLE 1

| Name | Peptide Sequence |
|---|---|
| Holotocin(HLT) | CFITNCPLG-NH2 (SEQ ID NO: 1) |
| Oxytocin(OXT) | CYIQNCPLG-NH2 (SEQ ID NO: 2) |
| Vasopressin(AVP) | CYFQNCPRG-NH2 (SEQ ID NO: 3) |
| Octopressin(OTP) | CFWTNCPVG-NH2 (SEQ ID NO: 4) |

### Preparation Example 2: Cell Culture

Epidermal keratinocytes HaCaT and kidney cell line 293T were seeded into 96-well plates at a density of 5×10³ cells per well, and cultured for 24 hours in a 5% CO₂ incubator at 37° C. The culture was performed using a commonly used method.

In the following Examples, the holotocin (HLT), oxytocin (OXT), vasopressin (AVP), and octopressin (OTP) peptides prepare in Preparation Example 1, and the epidermal keratinocytes HaCaT and kidney cell line 293T prepared in Preparation Example 2 were used.

### Example 1: Evaluation of Cytotoxicity of Marine Peptides in 293T and HaCaT Cells

A CCK-8 assay was performed to evaluate the cytotoxicity of the marine-derived peptides.

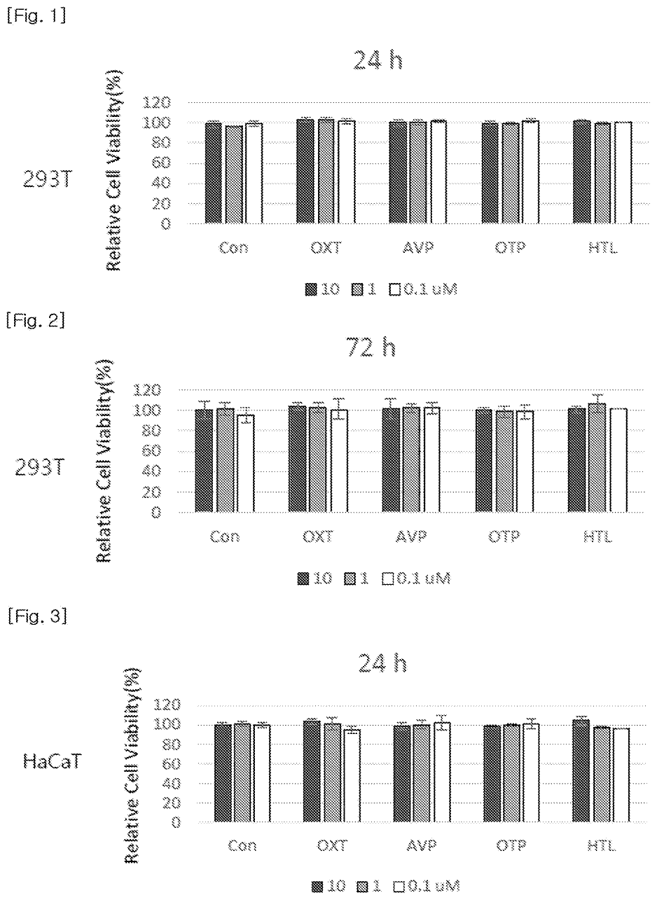

The peptides prepared in Preparation Example 1 were added to the cells prepared in Preparation Example 2 at concentrations of 10, 1, and 0.1 μM, respectively, and the cells were cultured for 24 and 72 hours. Subsequently, the cells were reacted in a medium containing 10 μL of CCK-8 solution for 3 hours, and the absorbance at 450 nm was measured using a spectrophotometer (Molecular Devices, SpectraMax i3x). The results are shown in FIGS. 1 to 4.

At this time, cell viability was calculated as the percentage of viable cells, and all results were repeated three times.

Referring to FIGS. 1 and 2, as a result of comparing cell viability in 293T cells, it was confirmed that there were no changes in cytotoxicity or cell proliferation under any of the conditions treated with HLT, OXT, AVP, or OTP.

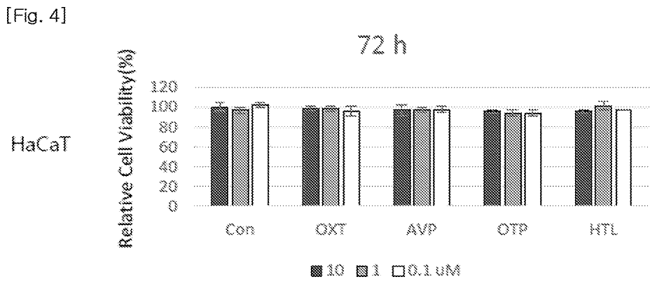

Referring to FIGS. 3 and 4, as a result of comparing cell viability in HaCaT cells, it was confirmed that there were no changes in cytotoxicity or cell proliferation under any of the conditions treated with HLT, OXT, AVP, or OTP.

Accordingly, it was found that the holotocin (HLT) peptide and the octopressin (OTP) peptide are safe natural substances exhibiting no cytotoxicity to cells, regardless of treatment concentration or duration.

### Example 2: Evaluation of the Size of Stem Cell Spheroids Formed in 293T and HaCaT Cells in Response to Marine Peptides

For the formation of spheroids, the cells prepared in Preparation Example 2 were cultured in Aggrewell 800 plates pretreated with an anti-Adherence rinsing solution, using a DMEM-F12 medium containing a stem cell-permissive medium supplemented with 20 ng/ml EGF, 20 ng/ml basic fibroblast growth factor (bFGf), and B27 Serum-free Supplement (50×; Invitrogen).

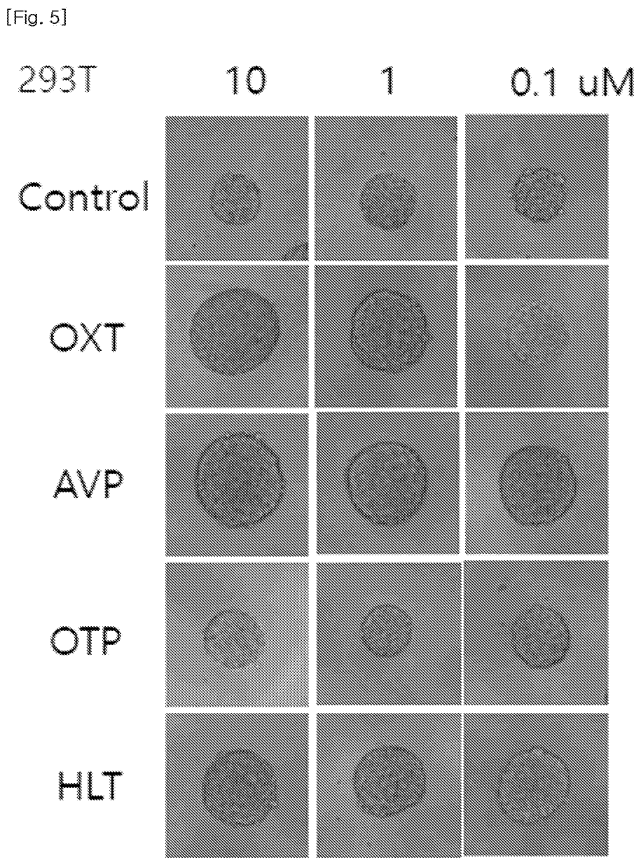

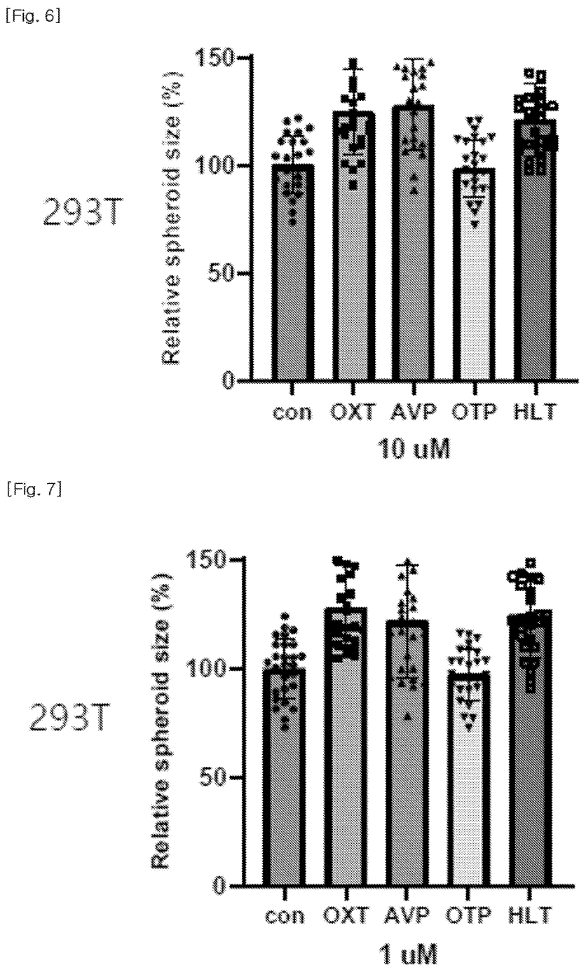

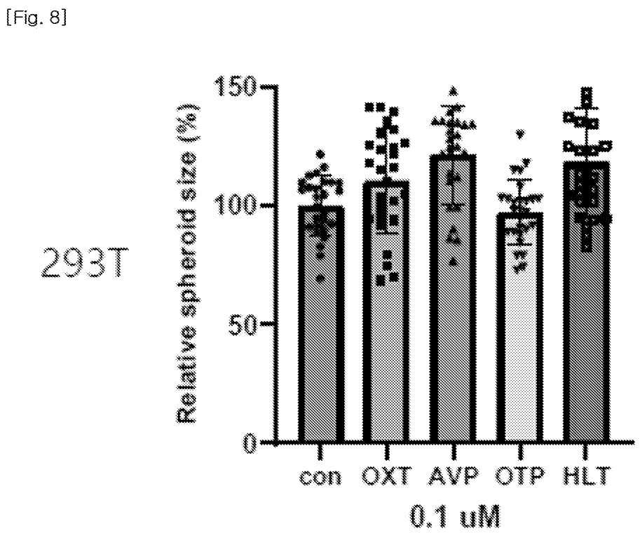

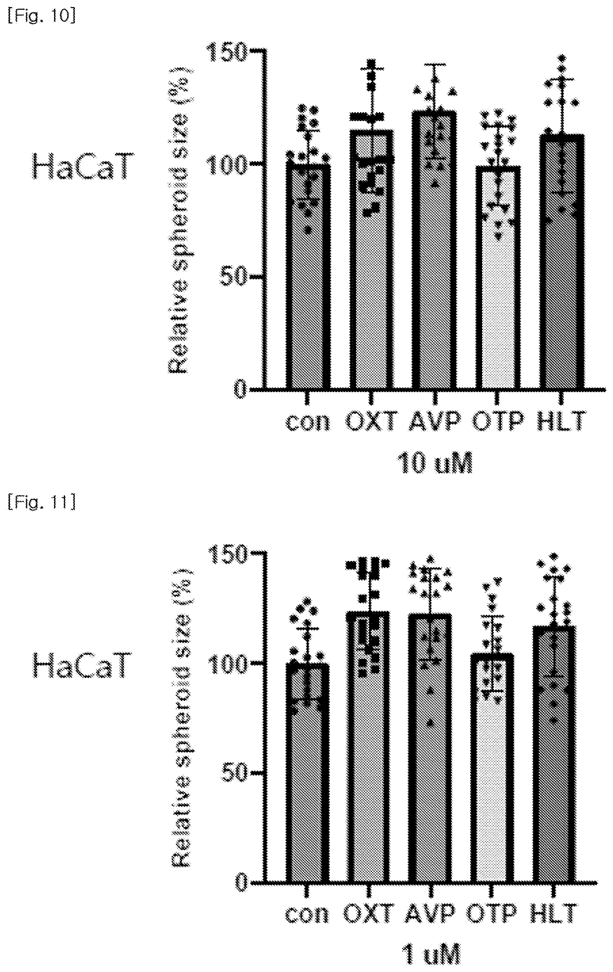

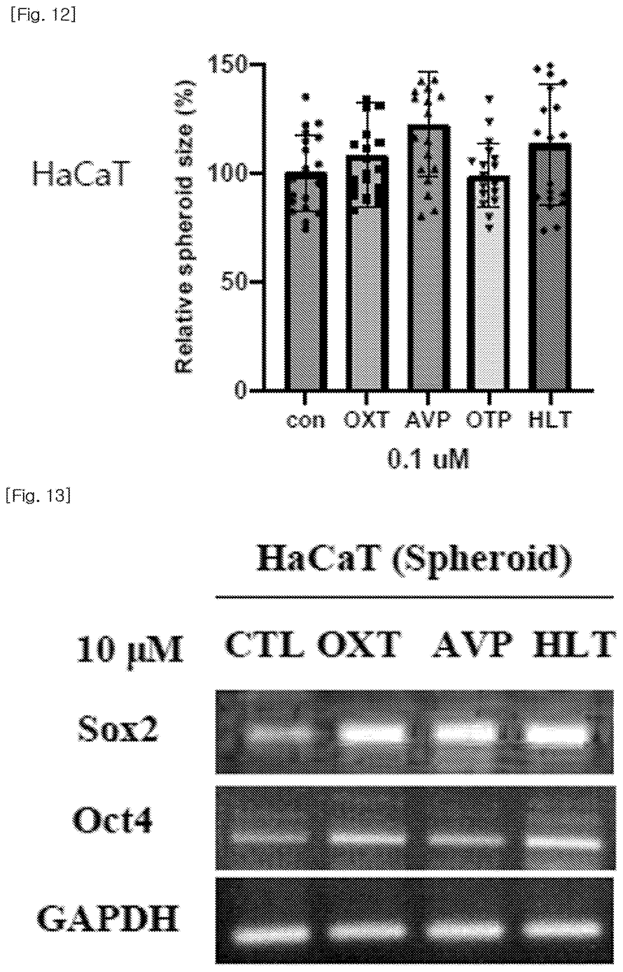

Subsequently, the peptides prepared in Preparation Example 1 were added at concentrations of 10, 1, and 0.1 μM, respectively, and the cells were cultured for 10 days. After the culture period, the spheroids were observed under a microscope, and approximately 25 spheroids were measured. The results are shown in FIGS. 5 to 12.

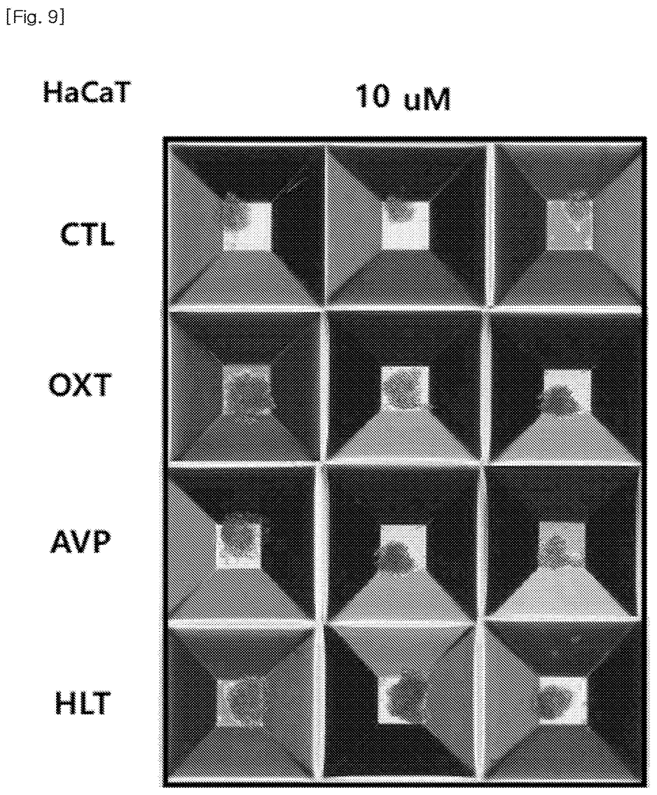

Specifically, FIGS. 5 to 8 show the results of adding the peptides prepared in Preparation Example 1 to the renal cell line 293T, and FIGS. 9 to 12 show the results of adding the same peptides to the epidermal keratinocytes HaCaT.

Referring to FIGS. 5 to 12, it was confirmed that treatment with the octopressin (OTP) peptide resulted in little or no change in the size of the stem cell spheroids compared to the control group.

In contrast, treatment with the holotocin (HLT) peptide was found to increase the size of the stem cell spheroids compared to the control group: in the kidney cell line 293T, the size increased by 121.4% (10 μM), 124.3% (1 μM), and 118.4% (0.1 μM); and in the epidermal keratinocytes, the size increased by 108.7% (10 μM), 122.1% (1 μM), and 117.5% (0.1 μM).

It was also confirmed that the increase in the size of the stem cell spheroids upon treatment with oxytocin (OXT) and vasopressin (AVP) peptides was similar to that observed with the holotocin (HLT) peptide.

Therefore, the oxytocin (OXT), vasopressin (AVP), and holotocin (HLT) peptides were found to promote the formation and/or increase the size of stem cell spheroids.

### Example 3: Expression of Regeneration-Related Genes in HaCaT in Response to Holotocin

### 1) Analysis of Regeneration-Related Gene Expression by Polymerase Chain Reaction (PCR)

To compare the expression levels of the cell regeneration-related genes in HaCaT skin cells, reverse transcription polymerase chain reaction (RT-PCR) was performed.

Specifically, oxytocin (OXT), vasopressin (AVP), and holotocin (HLT) peptides prepared in Preparation Example 1 were each applied at a concentration of 1 μM to HaCaT skin cells prepared in Preparation Example 2, and then cultured for 24 hours to promote the formation of spheroids. RNA was extracted from the HaCaT cells using TRIzol RNA extraction.

Next, reverse transcription polymerase chain reaction (RT-PCR) was performed on 1 μg of total cDNA synthesized using the Transcriptor First Strand cDNA Synthesis Kit (Roche, Germany). Subsequently, PCT was performed using each primer pair under the following conditions: pre-denaturation at 94° C. for 5 minutes; followed by 30 cycles of denaturation at 94° C. for 1 minute, annealing at 56° C. for 1 minute, and extension at 72° C. for 1 minute and 30 seconds; and a final extension at 72° C. for 5 minutes. The expression levels of each gene were confirmed based on the PCR results.

At this time, the primers of Sox2 and Oct4 listed in Table 2 below were used as stemness markers to determine changes in gene expression, and the results are shown in FIG. 13.

TABLE 2

| Primer Name | Direction | Sequence |
|---|---|---|
| Sox2 | forward | CAAGATGCACAACTCGGAGA |
| | | (SEQ ID NO: 5) |
| | reverse | TTCATGTGCGCGTAACTGTC |
| | | (SEQ ID NO: 6) |
| Oct4 | forward | TGGGATATACACAGGCCGAT |
| | | (SEQ ID NO: 7) |
| | reverse | GTGACAGAGACAGGGGGAAA |
| | | (SEQ ID NO: 8) |
| GAPDH | forward | GACAGTCAGCCGCATCTTCT |
| | | (SEQ ID NO: 9) |
| | reverse | GCGCCCAATACGACCAAATC |
| | | (SEQ ID NO: 10) |

### 2) Analysis of Regeneration-Related Gene Expression by Immunofluorescence

To compare the expression levels of the cell regeneration-related genes in HaCaT skin cells, immunofluorescence was performed.

Specifically, HaCaT skin cells prepared in Preparation Example 2 were cultured on cover glasses and treated with 1 μM of oxytocin (OXT), vasopressin (AVP), and holotocin (HLT) peptides prepared in Preparation Example 1 for 24 hours.

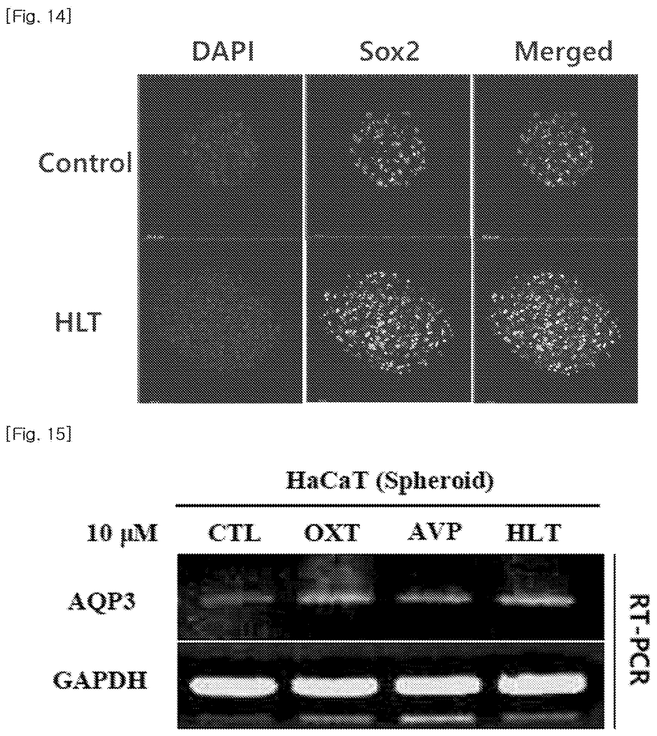

Subsequently, the cells were fixed with 4% formaldehyde, followed by treatment with 0.1% Triton X-100 to allow the antibodies to penetrate the cell membrane. The cells were then incubated with a primary antibody specific to SOX-2 (E-4; sc-365823) for 12 hours, followed by incubation with a secondary antibody conjugated with the red fluorescent dye Alexa Fluor 555 (Invitrogen) for 1 hour. Thereafter, the cover glasses were washed with PBS buffer for 10 minutes, followed by staining with DAPI (Invitrogen), a reagent that selectively binds to DNA. The stained samples were then observed under a fluorescence microscope, and the results are shown in FIG. 14.

Referring to FIG. 13, it was confirmed that treatment with the holotocin (HLT) peptide induced the expression of the stemness-related genes Sox2 and Oct4, which promote the formation of stem cell spheroids, at levels comparable to those observed with oxytocin (OXT) and vasopressin (AVP).

Referring to FIG. 14, it was confirmed that treatment with the holotocin (HLT) peptide led to an increased expression of the SOX-2 protein.

Therefore, it was found that the holotocin (HLT) peptide, similar to oxytocin (OXT) and vasopressin (AVP) peptides, induces the expression of cell regeneration-related genes (Sox2 and Oct4), exhibiting the effect of cell regeneration or regeneration promotion.

### Example 4: Expression of AQP3 Gene in HaCaT in Response to Holotocin

### 1) Analysis of AQP3 Gene Expression by Polymerase Chain Reaction (PCR) and Immunofluorescence

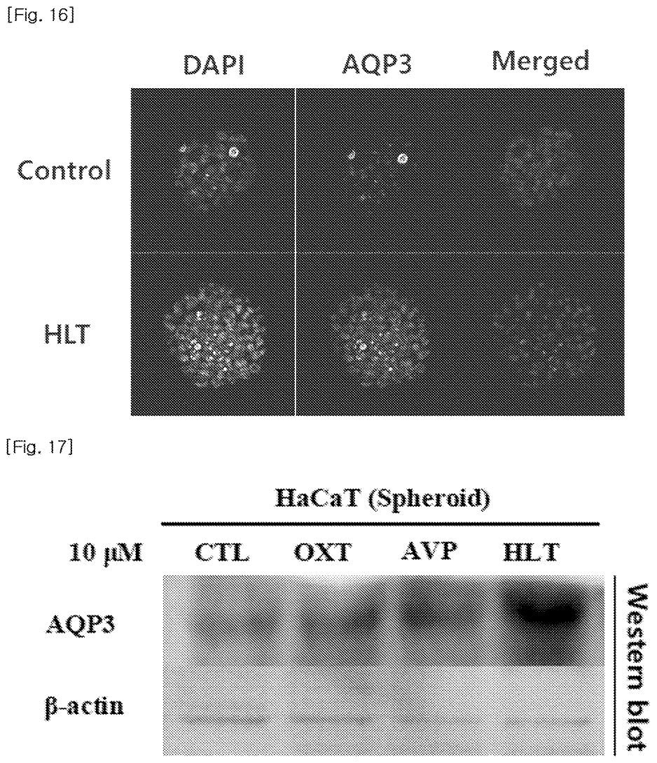

To compare the expression levels of the AQP3 gene in HaCaT skin cells, polymerase chain reaction (PCR) and immunofluorescence were performed in the same manner as in Example 3, and the results are shown in FIGS. 15 and 16.

At this time, the expression of the AQP3 gene was identified using the primers listed in Table 3 below.

TABLE 3

| Primer Name | Direction | Sequence |
|---|---|---|
| | forward | TGATGCAATCTGGCACTTCG |
| | | (SSQ ID NO: 11) |
| | reverse | GCCAGCACACACACGATAAG |
| | | (SEQ ID NO: 12) |

### 2) Analysis of Protein Expression by Western Blot Assay

To compare the expression levels of the AQP3 gene in HaCaT skin cells, a Western blot assay was performed.

Specifically, HaCaT cells were lysed using a protein lysis solution and then centrifuged at high speed to collect the cell lysates. The protein concentration in the cell lysates was then analyzed using the Pierce BCA Protein Assay Reagent (Thermo Scientific, Rockford, IL, USA). Subsequently, the samples containing 30 μg of total protein were subjected to sodium dodecyl sulfate-polyacrylamide gel electrophoresis (SDS-PAGE) to separate the proteins present in the cells. Next, to confirm protein expression, the proteins were incubated with AQP3 (F-1; sc-518001) and β-actin (C4; sc-47778) antibodies for 12 hours, followed by a 1-hour incubation with a mouse secondary antibody (Santa Cruz biotechnology) that recognizes the primary antibody. Changes in protein expression levels were then analyzed using an enhanced chemiluminescence (ECL) detection system (Amersham Pharmacia Biotechnology), and the results are shown in FIG. 17.

Referring to FIGS. 15 to 17, it was confirmed that treatment with the holotocin (HLT) peptide resulted in a slightly higher expression of the AQP3 gene compared to oxytocin (OXT) and vasopressin (AVP).

Therefore, referring to Examples 1 to 4 and FIG. 17, it was found that, according to the present invention, the treatment of cells with the holotocin peptide increased the size of stem cell spheroids without causing cytotoxicity, similarly to oxytocin (OXT) and vasopressin (AVP). Moreover, the expression of genes that promote the formation and/or increase the size of stem cell spheroids was observed, exhibiting the effect of cell regeneration or regeneration promotion. In addition, it was found that increased expression of the AQP3 gene exhibits excellent effects on wound healing, regeneration, skin moisturization, or anti-aging in the skin or other body tissues.

In summary, it was found that the holotocin peptide not only promotes the formation of stem cell spheroids and enhances wound-healing ability through its effect of cell regeneration or regeneration promotion, but also enhances the effect of skin cell regeneration, skin barrier function improvement, skin moisturization, or anti-aging.

Although certain embodiments of the present invention have been described in detail above, it will be apparent to those skilled in the art that such detailed descriptions are merely exemplary of preferred embodiments and should not be construed as limiting the scope of the present invention. Thus, the substantial scope of the present invention will be defined by the appended claims and equivalents thereof.

1. A composition for promoting the formation of stem cell spheroids, comprising a sea cucumber-derived holotocin peptide.

2. The composition for promoting the formation of stem cell spheroids according to claim 1, wherein the sea cucumber is Stichopus japonicus Selenka.

3. The composition for promoting the formation of stem cell spheroids according to claim 1, wherein the sea cucumber-derived holotocin peptide comprises the amino acid sequence of SEQ ID NO: 1.

4. The composition for promoting the formation of stem cell spheroids according to claim 1, wherein the sea cucumber-derived holotocin peptide increases the size of three-dimensional cell spheroids by 105% to 130% during cell culture.

5. The composition for promoting the formation of stem cell spheroids according to claim 1, wherein the sea cucumber-derived holotocin peptide increases the expression of at least one gene or protein selected from the group consisting of Sox2 (sex determining region Y-box 2), Oct4 (octamer-binding transcription factor 4), and AQP3 (Aquaporin 3).

6. The composition for promoting the formation of stem cell spheroids according to claim 1, wherein the sea cucumber-derived holotocin peptide promotes the regeneration of at least one cell selected from the group consisting of stem cells, hepatocytes, skin cells, muscle cells, fibroblasts, and nerve cells.

7. The composition for promoting the formation of stem cell spheroids according to claim 1, wherein the composition for promoting the formation of stem cell spheroids has the function of cell regeneration, skin regeneration, skin wound healing, skin barrier improvement, skin moisturization, or anti-aging.

8. A method for promoting the formation of stem cell spheroids, comprising the step of treating cells with a sea cucumber-derived holotocin peptide.

9. A pharmaceutical composition for cell regeneration or wound healing, comprising the composition for promoting the formation of stem cell spheroids according to claim 1.

10. A cosmetic composition for skin cell regeneration, moisturization, or anti-aging, comprising the composition for promoting the formation of stem cell spheroids according to claim 1.

11. A topical composition for cell regeneration or wound healing, comprising the composition for promoting the formation of stem cell spheroids according to claim 1.

12. A quasi-drug composition for cell regeneration or wound healing, comprising the composition for promoting the formation of stem cell spheroids according to claim 1.

13. A health functional food for cell regeneration or anti-aging, comprising the composition for promoting the formation of stem cell spheroids according to claim 1.

14. A feed composition for cell regeneration or anti-aging, comprising the composition for promoting the formation of stem cell spheroids according to claim 1.
